(19) United States
(12) Patent Application Publication
(10) Pub. No.: US 2026/0060544 A1
(43) Pub. Date: Mar. 5, 2026

(54) Death Notification Device, System and Method of Use

(71) Applicant: Anna Page Campbell, Heathsville, VA (US)

(72) Inventor: Anna Page Campbell, Heathsville, VA (US)

(21) Appl. No.: 18/817,653

(22) Filed: Aug. 28, 2024

(51) Int. Cl. A61B 5/00; A61B 5/0205; A61B 5/11

(52) U.S. Cl. CPC A61B 5/0022; A61B 5/0031; A61B 5/02055; A61B 5/1112; A61B 5/48; A61B 5/6803; A61B 5/6804; A61B 2560/0214

(57) ABSTRACT

A wearable or implantable microchip system for discerning death of a human or animal and the location of the body, and providing timely notice to designated, remotely located parties.

### CROSS REFERENCE TO RELATED APPLICATIONS

Not applicable

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

### Field of the Invention

The present invention relates to a micro-chip for notifying third parties about the death of a human or animal based on a change in one or more biomarkers or vital signs.

### Background of the Invention

Each year, many individuals pass away while at home alone and are not found by family or friends for long periods of hours or days. During these time periods, the remains of the deceased are vulnerable to interference by the elements and, if left long enough, even suffer from decay before being discovered.

Similarly, the life status of persons who have gone missing or been abducted against their will is often unknown to their loved ones. “Proof of life” is often an issue in negotiations related to abductions.

In senior citizens, especially those suffering from the onset of dementia, the occurrence of wandering from one's place of residence and becoming lost is quite common.

Similarly, authorities in many jurisdictions are monitoring the presence of various species of animals, for example endangered species in national parks.

Whether discerning the life status, vital statistics or location of a person is at issue, a solution is to provide loved ones and emergency responders with the ability to receive near-instantaneous notice of a change in status. What is needed is a device and method for providing timely notice to interested parties of any such change in status.

The death notification system disclosed herein below serves to monitor the user of the device/system for a change in status related to body temperature, vital signs or location, and provide an alert to interested parties to allow for timely intervention upon notification of death, preferably within an hour's time.

### BRIEF DESCRIPTION OF THE DRAWINGS

FIG. 1 is a line drawing showing an example of the inventive implantable or wearable device.

FIG. 2 is a line drawing showing the device of FIG. 1 attached to a wearable strap.

FIG. 3 is a line drawing showing a view of the device of FIG. 1 implanted in a human being.

FIG. 4 is a line drawing showing a system and method of use of the device of FIG. 1.

### DETAILED DESCRIPTION OF THE INVENTION

The invention is an alert or notification system for monitoring the body heat of a human person or animal to verify that such person or animal remains alive, which will only emit a signal/alert once the body heat of the monitored person or animal drops to a level indicating death. Alternative embodiments could involve the monitoring of heartbeat, blood pressure, or other vital signs indicating the death of the user. In another preferred embodiment, the temperature-monitoring function of the microchip system can be paired with one or more other useful functionality, such as a location indicator utilizing GPS.

The microchip system will integrate a microcontroller or microprocessor with peripherals such as a Wi-Fi module or antenna, or coprocessor. The microchip system may optionally include a built-in memory feature or wirelessly connect to an external memory.

In a preferred embodiment, the microchip system would be a system-on-a-chip device, an integrated circuit that integrates all components of a computer, notably a CPU, memory or memory interface, input/output devices and/or interfaces, optional secondary storage interfaces and radio modems or other transponders, all on a single substrate or microchip. The chip will comprise a sensor function for monitoring indicia of death, such as decreased temperature or absent magnetic or electrical fields.

In an alternate embodiment, the microchip system will be a system in a package. The microchip system may be paired with dedicated and physically separate memory and secondary storage, such as LPDDR and eUFS or eMMC chips, respectively, that may be layered on top of the chip as a package on package configuration. In one iteration, the chip may be paired with a separate wireless modem, for example on a cellphone, smartwatch or other wearable device.

Radio frequency functions on a substrate, and may comprise digital, analog or mixed-signal functions.

In one embodiment, the implantable microchip system employs the dielectric spectroscopy principle for detecting biomarkers in blood or interstitial fluid of a human or animal, wherein each biomarker is associated with an oscillation frequency which can be detected by a sensor. Frequency ranges of 10-100 GHz may be used for biomarker detection. More preferably, the sensor(s) will be embodied as terahertz sensors, operating at 100 GHz or above, optionally using Bipolar, CMOS or BiCMOS processes, allowing for better miniaturization of the chip.

Sensors comprised in the microchip system can be used to interact with a variety of soft body tissues as well as bodily fluids, including but not limited to blood, lymph, urine, saliva, tears and, if the wearable version of the system is employed, sweat.

In the preferred embodiment, the microchip system will not include a battery, but will instead draw power via a transponder from the user's body heat using a miniaturized thermoelectric generator (TEG). In such embodiment, the system may be arranged as a six-transistor or eight-transistor low-voltage or ultra-low voltage chip. Such embodiment may further utilize a plethora of TEGs. Such arrangements are now known in the field of pacemakers, body armor and other electronic medical devices surgically implanted within patients'bodies. TEGs employed in the microchip system will preferably use materials such as bismuth telluride (Bi2Te3) or lead telluride (PbTe), with a zT of 2-3.

In other embodiments, the system draws power from a group of sources, including but not limited to a small solar panel, an antenna that collects ambient radio wave energy, piezoelectric devices that collect energy from movement, or the radio-frequency energy emitted by the user's cell phone or other radio-emitting device(s).

The user may use the microchip system by locating it on or in a wearable item. Possible items would include, without limitation, a bracelet or other jewelry, a hair piece or extension, a patch or bandage, or an article of clothing such as a wristband or scarf. Alternatively, the chip could be located subcutaneously, injected or otherwise placed within the user's body.

The microchip system should be encased in a water-resistant cover or casing, preferably made of durable plastic or rubber.

Upon the user's body temperature dipping below a set level indicating death, the device microprocessor would be programmed to activate the radio emission function, sending a radio signal indicating the death of the user. In another embodiment, the programming would activate such a signal upon the user ceasing to emit body temperature.

In practice, the microchip system forwards an alert to a central processor, which is itself programmed to forward the alert to one or more predetermined persons for notification. Such alert may be transmitted by any known radio transmission method, including but not limited to satellite transmission and Wi-Fi transmission, perhaps comprising an antenna. As such, notice parties are notified within seconds or moments of an alert-causing change, allowing for quick response. The microchip system's notice may also trigger a response from a dedicated, human operator, to first call the user to confirm a status change, either by telephone call or by the user's failure to answer such a call, and follow up with second calls to family members, neighbors and/or emergency responders. In one embodiment, the user or account holder for the user's chip would pre-select notice parties.

In one embodiment, alerts or signals emitted by the microchip system would be provided as an Emergency Position-Indicating Radio Beacon, or EPRB. In another, the signal or alert would notify parties holding relevant documents or account information regarding the user (e.g., will, financial and medical records, etc.) to release such documents or information to parties prechosen by the deceased user. In another, the device comprises a RFID component as a means of passive tagging and tracking.

In another preferred embodiment, the inventive device is a passive system which is dormant (inactive) until death of the wearer. In another, upon the death of the human or animal being monitored by the device, the device activates then emits a signal that alerts the closest EMT office and the wearer's chosen ICE contact of the deceased′ body exact location for physical recovery purposes.

### LIST OF COMPONENTS

10 Microchip System
20 Sensors
21 Thermal
22 Pressure
23 Electrical
24 Magnetic
30 Transponder
40 CPU/Ram/Storage
50 Power Supply
60 Memory/memory interface
90 Wearable article
100 External transponder
110 Internet
120 Third party monitoring device

### DETAILED DESCRIPTION OF THE DRAWINGS

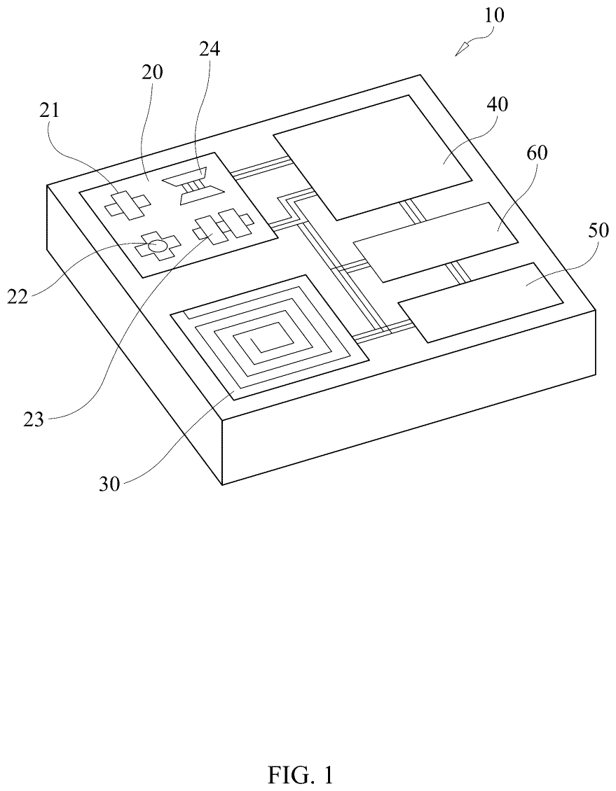

Turning to the figures, FIG. 1 is a schematic representation of one embodiment of the inventive implantable or wearable microchip system 10. The device is embodied as a system-on-a-chip, showing components sensors 20, transponder 30, CPU with ram and/or storage 40 and a power supply 50. For purposes of illustration, the sensor component includes four types of sensors: thermal 21, pressure 22, electrical 23 and magnetic 24. Depending upon the placement of the device on or in the user's body, whether on the skin via a wearable device, subcutaneous or interstitial arrangement, or arrangement next to a blood vessel, one or more of the integrated sensors will be able to actively monitor a relevant biomarker indicating whether the user is alive or dead. An optional position locator is not pictured, but could be used as a locator for the receiver of the transmitted signals to find and retrieve the user's body as needed.

In certain embodiments, the memory is a solid state drive, the transponder is a miniaturized antenna or similar device and the power supply may be a conduit for drawing power from a second device, such as a cell phone, or a kinetic power generator. In a preferred embodiment, the power supply is a thermoelectric generator, creating electric power from the user's body heat.

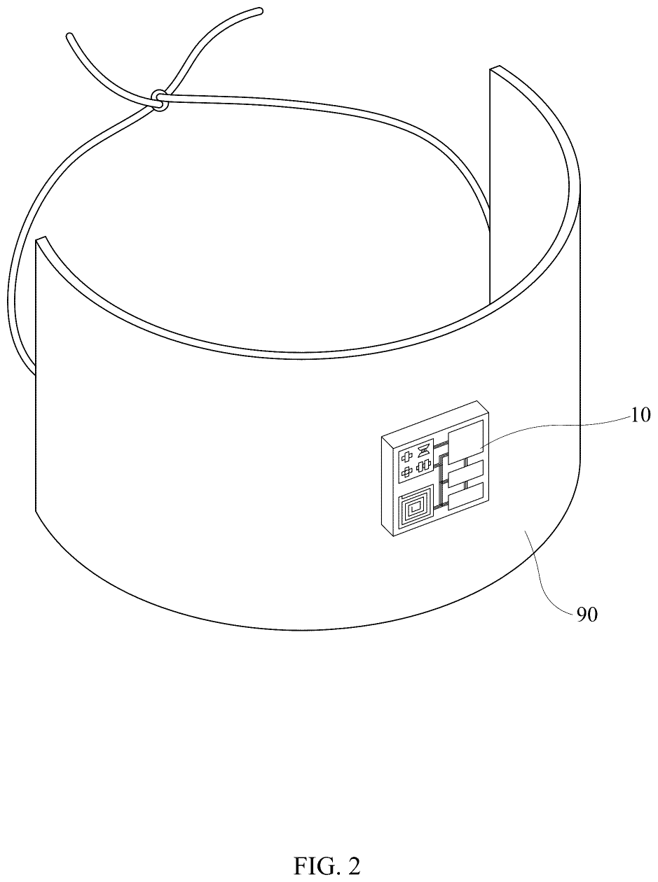

FIG. 2 shows the inventive microchip system 10 embedded into a wearable article 90, here shown as a bracelet or collar with adjustable straps such that the chip can be brought into contact with the skin of a user's arm, leg, wrist, torso, neck or other body part.

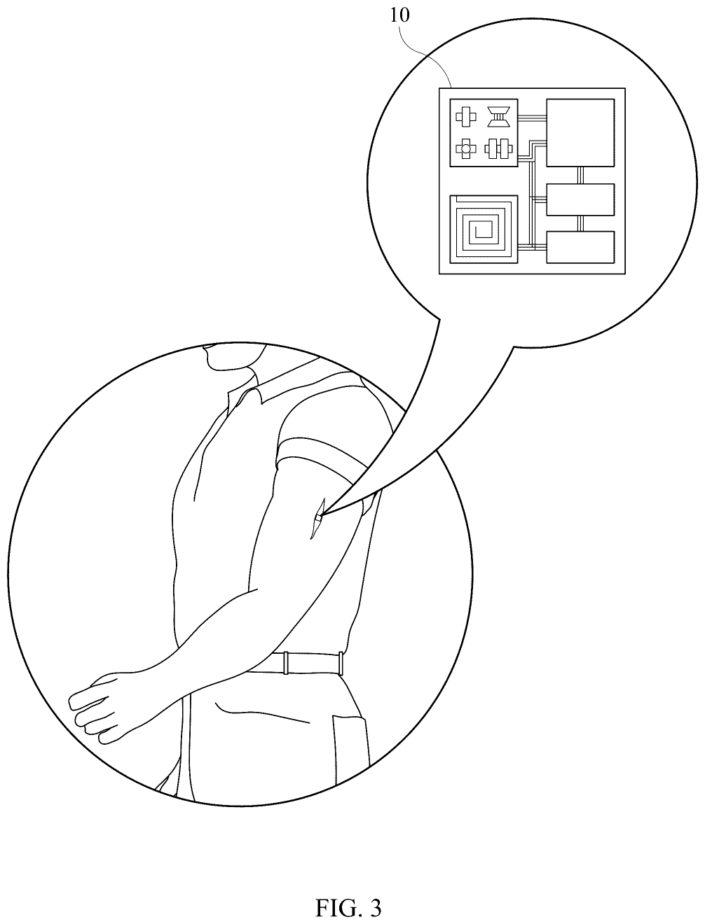

FIG. 3 shows the inventive microchip system 10 surgically implanted into a user's arm via an incision. Such surgical implantation will allow for multiple types of internal placement of the system, whether it be subcutaneous, in interstitial tissue, or adjacent to a blood vessel or organ, depending upon the types of sensors to be employed.

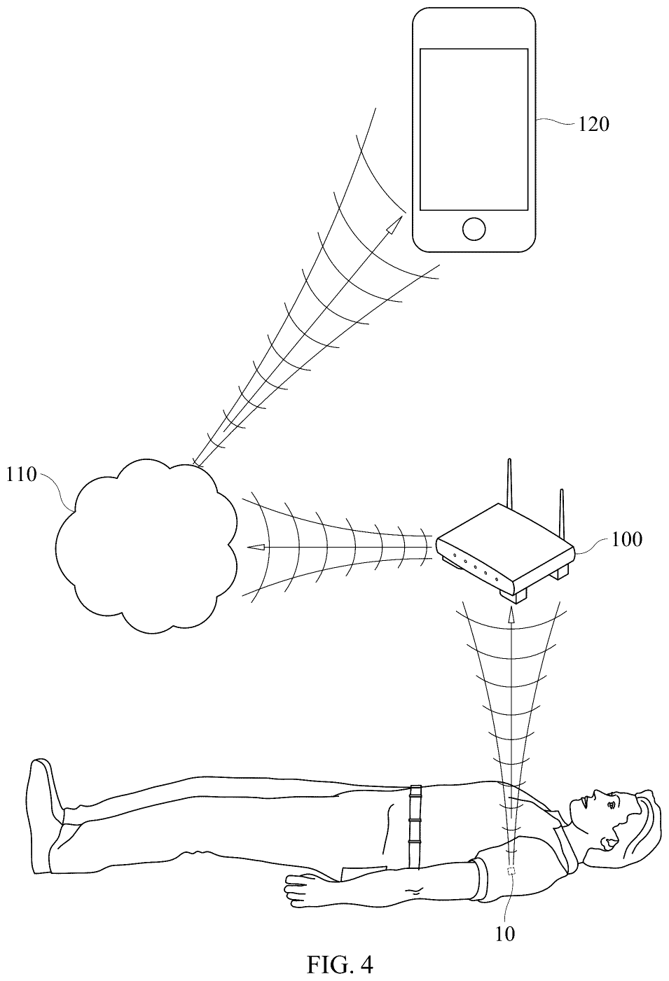

FIG. 4 shows the inventive microchip system 10 after implantation and in use, emitting a signal to an external transponder 100, to the internet 110 and thence to a third party monitoring device 120. In the preferred example, the signal will inform the owner of the third party monitoring device of the user's death based on one or more biomarker readings of the system sensors 20 (not pictured).

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

I claim:

1. A wearable or implantable microchip system, wherein the system comprises one or more sensors which monitors one or more of a user's biomarkers, comprising body temperature, blood circulation, blood-oxygen level, electric current, magnetic field and/or respiration, optionally together with the physical location, wherein, upon such sensor(s) measuring a predetermined change in one or more biomarkers, such sensor(s) signal a radio transponder component to emit a notice signal indicating the death of the user.

2. The microchip system of claim 1, wherein, upon a predetermined change in any measured biomarker, the system signals a radio transponder component, which transponder emits a notice signal indicating such change in vital sign(s).

3. The microchip system of claim 1, wherein the system lacks a battery and instead draws power from the user's body heat and/or movement.

4. The microchip system of claim 1, wherein the system lacks a battery and instead draws power from the radio emissions of a paired cellular phone or similar electronic device(s).

5. The microchip system of claim 1, wherein the microchip system is a system-on-a-chip and comprises a central processing unit, a storage or storage interface, a memory or memory interface, a transponder, one or more sensors, and a power supply.

6. The microchip system of claim 5, wherein the power supply is a thermoelectric generator.

7. The microchip system of claim 1, wherein the system also provides notice of the user's geographic location.

8. The microchip system of claim 1, wherein the microchip system is arranged on or in a piece of wearable jewelry, clothing, patch or bandage so that the microchip system is touching the user's skin.

9. The microchip system of claim 1, wherein the microchip system is arranged on or in a hair clip or hair extension so that the microchip system is touching the user's skin.

10. The microchip system of claim 1, wherein the transponder is a Wi-Fi module.

11. The microchip system of claim 1, wherein the transponder is a satellite transmitter.

12. The microchip system of claim 1, embodied as a system in a package.

13. A method of using the microchip system of claim 1, comprising the steps of
a. injecting or surgically inserting the microchip system into a user's body;
b. placing the microchip system subcutaneously;
c. monitoring one or more user biomarkers with sensor components of the microchip system, and
d. signaling the user's death upon the microchip system measuring one or more biomarkers indicating death.

14. The method of claim 13, replacing step b with:
b. placing the microchip system in the user's interstitial tissue.

15. The method of claim 13, replacing step d with:
d. a sensor monitoring a biomarker indicating death signaling the user's death to a radio transponder;
e. emitting the signal from the radio transponder to an external transponder; and
f. optionally, forwarding the signal from the external transponder to one or more third parties via a connection between the external transponder and the internet.
